(19) United States
(12) Patent Application Publication
(10) Pub. No.: US 2004/0014214 A1
(43) Pub. Date: Jan. 22, 2004

(54) Method for producing a recipient-specific tissue transplant

(76) Inventor: Augustinus Bader, Immensen (DE)

Correspondence Address: CLARK & ELBING LLP, 101 FEDERAL STREET, BOSTON, MA, 02110, US

(21) Appl. No.: 10/296,674

(22) PCT Filed: May 28, 2001
(86) PCT No.: PCT/DE01/01985

(30) Foreign Application Priority Data
May 29, 2000 (DE) 100264425

(51) Int. Cl. C12N 5/08

(52) U.S. Cl. 435/401; 623/915

(57) ABSTRACT

The invention relates to a method for the production of a recipient-specific tissue transplant, consisting of a tissue matrix and recipient-acceptable cells populating said matrix. The transplant prepared and intended for populating is sterilized with hydrogen peroxide. Sterilization with hydrogen peroxide reliably prevents contamination while preserving the tissue base.

The invention relates to a method for preparing a recipient-specific tissue transplant composed of a tissue matrix, i.e. a biological tissue which is intended for the transplantation and recipient-tolerated cells which have been applied to it.

The method can be applied generally to methods for the controlled culture or biological tissue using autologous, allogenic or xenogenic starting materials.

In transplantation medicine, there is a great need for suitable transplants which induce adverse reactions in the transplant recipient to the smallest possible extent. Only in certain cases is it possible to remove the transplant from the body of the recipient an graft it back into the recipient. While these transplantations are the safest from the immunological point of view, it is not possible to carry out such transplantations in the case of certain blood vessels or organs or when it is a matter of replacing relatively large areas of the skin. At present, only allogenic transplants from foreign donors or, frequently in the orthopedic field, synthetic implants made from plastics, metals, ceramic, etc., or from various composite materials, are in practice suitable for certain organs. When allogenic materials, such as donor organs, are used, constant immunosuppression, which is stressful for the body of the recipient, is required. Nevertheless, rejection reactions frequently occur as serious complications. Artificial materials can also give rise to rejection reactions and inflammatory processes, which ruin the surgical result.

For various reasons, frequent attempts are nowadays made to use xenogenic material (of animal origin). The particular advantage of this is the greater availability of this material as compared with allogenic (donor) materials. Furthermore, such a “biological material” is more flexible than an artificial material and adapts better to the recipient body at a number of sites. However, xenogenic transplantation material is problematical as a result of being strongly antigenic.

Attempts have therefore been made, for a relatively long time now, to make xenogenic and allogenic transplantation materials, especially different tissues which are earmarked for a transplantation, recipient-tolerated. For this, (allogenic or xenogenic) tissues which have been prepared are frequently colonized, in a suitable manner, with the recipient's own cells, i.e. autologous cells, or with recipient-tolerated, and frequently genetically altered, allogenic or xenogenic cells. This results in tissues which have been especially postcultured for the recipient.

In this connection, difficulties are frequently caused by the fact that the recipient-tolerated cells grow once again on the basic matrix in a manner which resembles the natural manner. Another problem is that xenogenic or allogenic starting materials may be infected with disease organisms (viruses or bacteria) which are new and, possibly, also foreign, to the recipient, such that the transplant brings with it dangers and stresses which the recipient does not require.

The object of the invention is therefore to design a method of the type mentioned at the outset such that the tissue matrix (tissue substratum) which is intended for the transplantation is optimally prepared for colonization with recipient-tolerated cells.

In order to achieve this object, the generic method is further developed, according to the invention, such that a sterilization with H₂O₂, preferably a plasma ionization with H₂O₂, is carried out for the purpose of preparing the tissue matrix prior to colonizing it with the recipient-tolerated cells.

In a further development of the invention, it is possible to add on further sterilization steps, such as treatment with detergent, γ-irradiation, gassing with ethylene oxide or enzymic treatment for killing the cells.

In addition, the tissue can be rinsed once or several times before or after the sterilization.

For the rest, the colonization with recipient-tolerated cells can be carried out using the methods which are known for this purpose, and customary, in the prior art, and which are merely extended by the sterilization step according to the invention.

Recipient-specific cells are generally speaking understood as being cells which are autologous for the recipient or which are immunologically compatible and/or tolerated as far as the recipient is concerned. It is also possible for different types of cells to be fed in at different colonization and/or treatment times, insofar as this is known in the prior art.

It has now been surprisingly found that a sterilization with H₂O₂ in particular, and particularly a plasma ionization with H₂O₂, prepares the underlying tissue optimally for subsequent colonization with the recipient's own cells or with recipient-tolerated cells. The sterilization hardly gives rise to any harmful byproducts at all. Contamination of the transplant, which is disadvantageous for the recipient, is prevented. Growing the cells which are envisaged for the colonization is made easier on the transplant which has been prepared in accordance with the invention.

In principle, any body cells are suitable for use as recipient-tolerated cells, including, for example, and depending on the underlying substratum, the following: connective tissue cells (inter alia fibroblasts and fibrocytes), muscle cells (myocytes), endothelial cells, skin cells (inter alia keratinocytes), cells (heart cells, kidney cells, etc.) which have differentiated into organ cells, preferably in the case of structured organs having a collagen scaffolding, and, in a general manner, any cells which can usefully be suggested for remodeling a particular tissue which is intended for the implantation. The precursor cells, preferably composed of autologous stem cells derived from the recipient, are also suitable. The stem cells include those which have already been mentioned above.

Prior to the treatment with the recipient-tolerated cells, the autologous, allogenic or xenogenic tissue which is intended for the transplantation, and which is present in native form or which is acellularized, is sterilized with H₂O₂ in accordance with the invention.

The invention has particular advantages in the case or xenogenic tissues, in connection with which a sterilization should in any case take place s: as to rule out with certainty any possibility of foreign viruses and bacteria being cointroduced into the freshly generated bioartificial transplant. It has been found that, while being outstandingly well tolerated by the substrate, that is the transplant which in to be colonized, the sterilization with H₂O₂ according to the invention leads to particularly good (reliable) results.

Any transfer of disease should also be ruled out with certainty in the case of allogenic starting tissues; the sterilization according to the invention is particularly advantageous in this case as well.

A sterilization by plasma ionization, in which a gaseous discharge takes place in the presence of H₂O₂, is at present regarded as being particularly suitable. For this, an aqueous solution of hydrogen peroxide is injected into a sterilization chamber and vaporized. Under reduced environmental pressure, a low temperature plasma is applied using a radio frequency energy. This thereby generates an electric field which produces a plasma. In the plasma state, the hydrogen peroxide is cleaved with the formation of free radicals. The free radicals constitute the active species for the sterilization.

The method according to the invention does not leave behind any toxic residues since, after the conclusion of the reaction, the free radicals react to give water, oxygen and other nontoxic products. The use of peroxides also corresponds to a natural process which occurs in many cells (for example in macrophages).

Where appropriate, the success of the sterilization can be specifically tested by, for example, testing, after the sterilization, for the presence of particular viruses or bacteria which should be completely prevented.

After it has been prepared, and prior to any possible sterilization which may be necessary, the tissue which is envisaged for the transplantation can be subjected to additional nondenaturing procedural steps. Rinsing once or several times before and/or after the sterilization is regarded as being advantageous.

After it has been colonized, the transplant can be subjected to further treatment, in a customary manner, or else immediately transferred to the site of the operation.

### EXAMPLE 1

Preparing Collagen Structures of Animal Origin for Implantations

Up until now, a collagen matrix has usually been sterilized, in an aqueous solution at pH 3.8 and in a glass receptacle, using chemical reagents such as chloroform. This chloroform evaporates, with continuous stirring at 4° C., over a period of 48-72 hours. After that, it is necessary to wait for up to 2 weeks for residues, which still have a cytotoxic effect, to evaporate off.

In accordance with the invention, the collagen solution is appropriately treated with H₂O₂ in greatly diluted form (1% or 0.1%) while continuously stirring at 4° C. An advantage is that it is possible to use the collagen sooner, i.e. after only 48 hours. It is likewise possible to subject the solution to plasma ionization. For this purpose, this process is carried out in a receptacle which is accessible to gas, with it being possible, in the ideal case, for the thickness of the medium bed to be up to 3 cm. In this connection, the plasma ionization process leads to an incipient crosslinking of the collagen and thus to stabilization of its shape. The subsequent colonization of these structures with porcine and human fibroblasts anid liver cells has proved to be particularly successful since both adhesion processes and migration processes within the matrix are facilitated.

### EXAMPLE 2

Preparing Collagen Structures of Animal Origin for Implantations

Skin or heart valve structures composed of native matrix are treated with an 0.1% solution of H₂O₂. The treatment process is effected by immersing the structures in the H₂O₂ solution for 15 minutes and subsequently washing them with sterile sodium chloride solution. The process can be repeated several times. The result is disinfection with an accompanying improvement in the ability of the implant structures to be colonized.

### EXAMPLE 3

Sterilizing Structured Collagen Matrices in Bioreactors

Bioreactors made of plastics, which contain or with plastic moldings, are sterilized, together with the materials they contain, for example collagen tile, as a complete unit. What are termed flat membrane bioreactors (FMBs) or rotatory bioreactors are suitable for this purpose. The flat membrane bioreactors are coated with collagen solutions, for example, by a portion of a 1.1% solution of collager being introduced and distributed together with a 10-fold concentrate of DMEM (Dulbecco's modified Eagle medium). After that, the bioreactors are treated with H₂O in the ionization chamber. This sterilization process results in an improvement in the stability of the coating, and to an increase in the internal stability of the implants and an improvement in the ability of the implants to be colonized.

### EXAMPLE 4

Sterilizing Tissue Fragments Covered of Native Matrix in Bioreactors

In accordance with example 2, the structures which are envisaged for the implantation can also be treated after having been fixed or having been introduced into bioreactors. Skin tissue fragments, or, for example, cardiovascular structures such as blood vessels or patches, are fixed in bioreactors and subsequently introduced into the plasma ionization chamber without any further treatment. In the chamber, they are treated cyclically with H₂O₂ plasma and after that recolonized directly, without any further treatment, in these bioreactors. Isolation and colonization protocols are used for this purpose.

1. A method for preparing a recipient-specific tissue transplant composed of a tissue matrix and recipient-tolerated cells which have colonized it,
characterized in that a sterilization with H₂O₂ is carried out for the purpose of preparing the tissue matrix prior to colonizing it with the recipient-tolerated cells.

2. The method as claimed in claim 1, characterized in that the sterilization is a plasma ionization with H₂O₂.

3. The method as claimed in claim 1 or 2, characterized in that a further sterilization, preferably treatment with detergent, γ-irradiation, gassing with ethylene oxide or enzymic treatment, is carried out in addition.

4. The method as claimed in one of claims 1 to 3, characterized in that the tissue which is intended for transplantation is additionally rinsed, once or several times, after it has been prepared, preferably before or after the sterilization.
